(19) United States
(12) Patent Application Publication
(10) Pub. No.: US 2025/0221667 A1
(43) Pub. Date: Jul. 10, 2025

(54) MONITORING AUXILIARY DEVICE

(71) Applicant: Largan Health AI-Tech Co., Ltd., Taipei City (TW)

(72) Inventors: Cheng-Kuo LAI, Taipei City (TW); Yuan-Yen YAO, Taipei City (TW); Chen-Hsiang CHEN, Taipei City (TW); Yu Jie HONG, Taipei City (TW); Chun-Hung TENG, Taipei City (TW)

(21) Appl. No.: 18/985,476

(22) Filed: Dec. 18, 2024

(30) Foreign Application Priority Data
Jan. 10, 2024 (TW) 113100981

(51) Int. Cl. A61B 5/00

(52) U.S. Cl. CPC A61B 5/6828; A61B 5/6831; A61B 5/4806

(57) ABSTRACT

A monitoring auxiliary device includes an outer surface layer, an inner surface layer, a middle layer, at least two electrode points, at least two conducting wires and at least two conducting sheets. The middle layer is located between the outer surface layer and the inner surface layer. The electrode points include a first electrode point and a second electrode point. The conducting wires include a first conducting wire and a second conducting wire. The conducting sheets include a first conducting sheet and a second conducting sheet. The electrode points are disposed on the outer surface layer of the monitoring auxiliary device, the conducting sheets are disposed on the inner surface layer of the monitoring auxiliary device.

### RELATED APPLICATIONS

This application claims priority to Taiwan Application Serial Number 113100981, filed Jan. 10, 2024, which is herein incorporated by reference.

### BACKGROUND

### Technical Field

The present disclosure relates to a monitoring auxiliary device. More particularly, the present disclosure relates to a monitoring auxiliary device applicable to sleeping monitoring.

### Description of Related Art

Monitoring device can help users understand the status of physiological functions, but traditional monitoring device require wires to be adhered to multiple places on the user's body, resulting in a poor wearing experience. Although the monitoring device has evolved that only need to be adhered to a specific part of the user, the monitoring device is still limited in size and weight, and is easy to fall off during wearing, resulting in poor monitoring results. Both of the aforementioned shortcomings will reduce users' willingness to wear the monitoring device. To increase users' willingness to purchase the monitoring device, it is necessary to actively develop wearing solutions that combine comfort and accuracy.

### SUMMARY

According to one aspect of the present disclosure, a monitoring auxiliary device, which is a sleeping monitoring auxiliary device, the sleeping monitoring auxiliary device is a wearable item, and the wearable item is a lower limb wearable item. The monitoring auxiliary device includes an outer surface layer, an inner surface layer, a middle layer, at least two electrode points, at least two conducting wires and at least two conducting sheets. The middle layer is located between the outer surface layer and the inner surface layer. The at least two electrode points include a first electrode point and a second electrode point. The at least two conducting wires include a first conducting wire and a second conducting wire. The conducting sheets include a first conducting sheet and a second conducting sheet. The at least two electrode points are disposed on the outer surface layer of the monitoring auxiliary device, and the at least two conducting sheets are disposed on the inner surface layer of the monitoring auxiliary device, the first conducting wire is connected to the first electrode point and the first conducting sheet, the second conducting wire is connected to the second electrode point and the second conducting sheet. When a maximum interval between the at least two conducting sheets is CDmax, and a monitoring auxiliary device width of the monitoring auxiliary device is Wa, the following condition is satisfied: 0.60≤CDmax/Wa.

According to another aspect of the present disclosure, a monitoring auxiliary device, which is a wearable item, and the monitoring auxiliary device includes at least two electrode points, at least two conducting wires and at least two conducting sheets. The at least two electrode points include a first electrode point and a second electrode point. The at least two conducting wires include a first conducting wire and a second conducting wire. The conducting sheets include a first conducting sheet and a second conducting sheet. The first conducting wire is connected to the first electrode point and the first conducting sheet, the second conducting wire is connected to the second electrode point and the second conducting sheet. When a maximum interval between the at least two conducting sheets is CDmax and a monitoring auxiliary device width of the monitoring auxiliary device is Wa, the following condition is satisfied: 0.70≤CDmax/Wa.

### BRIEF DESCRIPTION OF THE DRAWINGS

The present disclosure can be more fully understood by reading the following detailed description of the embodiment, with reference made to the accompanying drawings as follows:

FIG. 1 is a perspective view of a monitoring auxiliary device according to the 1 st embodiment of the present disclosure.

FIG. 2 is a top view of the monitoring auxiliary device according to the 1st embodiment of FIG. 1.

FIG. 3A is a schematic view of a breathable structure of the conducting sheet being a rectangular air vent.

FIG. 3B is a schematic view of a breathable structure of the conducting sheet being an oblique air vent.

FIG. 3C is a schematic view of a breathable structure of the conducting sheet being a mesh air vent.

FIG. 4 is a schematic view of a first electrode point and conducting sheet interval of the 1st embodiment of FIG. 1.

FIG. 5 is a perspective view of a monitoring auxiliary device according to the 2nd embodiment of the present disclosure.

FIG. 6 is a top view of the monitoring auxiliary device according to the 2nd embodiment of FIG. 5.

FIG. 7 is a schematic view of a first conducting sheet interval, a second conducting sheet interval and a third conducting sheet interval according to the 2nd embodiment of FIG. 6.

FIG. 8 is a perspective view of a monitoring auxiliary device according to the 3rd embodiment of the present disclosure.

FIG. 9 is a top view of the monitoring auxiliary device according to the 3rd embodiment of FIG. 8.

FIG. 10 is a perspective view of a monitoring auxiliary device according to the 4th embodiment of the present disclosure.

FIG. 11 is a perspective view of a monitoring auxiliary device according to the 5th embodiment of the present disclosure.

### DETAILED DESCRIPTION

A monitoring auxiliary device of the present disclosure is a sleeping monitoring auxiliary device, the sleeping monitoring auxiliary device is a wearable item, and the wearable item is a lower limb wearable item. The monitoring auxiliary device includes an outer surface layer, an inner surface layer, a middle layer, at least two electrode points, at least two conducting wires and at least two conducting sheets. The middle layer is located between the outer surface layer and the inner surface layer. The at least two electrode points include a first electrode point and a second electrode point. The at least two conducting wires include a first conducting wire and a second conducting wire. The at least two conducting sheets include a first conducting sheet and a second conducting sheet. The at least two electrode points are disposed on the outer surface layer of the monitoring auxiliary device, and the at least two conducting sheets are disposed on the inner surface layer of the monitoring auxiliary device, the first conducting wire is connected to the first electrode point and the first conducting sheet, the second conducting wire is connected to the second electrode point and the second conducting sheet. Thus, by connecting the electrode points and the monitoring device, attaching the conducting sheets with the user's skin, and connecting the conducting wires and the conducting sheets, it is favorable for extending the detection point of the monitoring device to the conducting sheets, and the monitoring device with larger size does not need to be adhered to the user's skin so as to improve the comfort of wearing the monitoring device.

Further, the at least two electrode points further include a third electrode point; the at least two conducting wires further include a third conducting wire; the at least two conducting sheets include a third conducting sheet. Thus, by increasing the number of the conducting sheets, the conducting wires and the electrode points, it is favorable for increasing the detection points of the monitoring device so as to improve the accuracy of the monitoring device.

When a maximum interval between the at least two conducting sheets is CDmax, and a monitoring auxiliary device width of the monitoring auxiliary device is Wa, the following condition is satisfied: 0.60≤CDmax/Wa. Thus, by satisfying the ratio of the maximum interval between the conducting sheets and the monitoring auxiliary device width of the monitoring auxiliary device, it is favorable for providing enough distance between the conducting sheets to ensure the monitoring current flows widely through the body so as to improve the accuracy of the monitoring device. Moreover, the following condition can be satisfied: 0.70≤CDmax/Wa. Further, the following condition can be satisfied: 0.76≤CDmax/Wa≤1.40. Moreover, the following condition can be satisfied: 0.90≤CDmax/Wa≤1.20. Further, the following condition can be satisfied: 0.96≤CDmax/Wa≤1.04.

When the maximum interval between the at least two conducting sheets is CDmax, the following condition is satisfied: 15 cm≤CDmax. The potential errors are easy to occur since the conducting sheets are too close. Thus, by satisfying the maximum interval between the conducting sheets, it is favorable for not only improving the monitoring accuracy, but also monitoring a variety of data required by the conducting sheets at long distances so as to increase the diversity of monitoring categories. Moreover, the following condition can be satisfied: 7.5 cm≤CDmax. Further, the following condition can be satisfied: 10 cm≤CDmax. Moreover, the following condition can be satisfied: 20 cm≤CDmax. Further, the following condition can be satisfied: 25 cm≤CDmax. Moreover, the following condition can be satisfied: 30 cm≤CDmax. Further, the following condition can be satisfied: 50 cm≤CDmax.

When a maximum length of the conducting sheets is LCmax, and the monitoring auxiliary device width of the monitoring auxiliary device is Wa, the following condition is satisfied: 0.05≤LCmax/Wa. Thus, by designing the appropriate ratio of the maximum length of the conducting sheets and the monitoring auxiliary device width of the monitoring auxiliary device, it is favorable for ensuring each of the conducting sheets with a sufficient proportion in the monitoring auxiliary device so as to increase the intensity of the potential signal. Moreover, the following condition can be satisfied: 0.07≤LCmax/Wa. Further, the following condition can be satisfied: 0.08≤LCmax/Wa≤0.20. Moreover, the following condition can be satisfied: 0.10≤LCmax/Wa≤0.15.

When the maximum length of the conducting sheets is LCmax, the following condition is satisfied: 2.50 cm≤LCmax≤5 cm. Thus, by designing each of the conducting sheets with an appropriate maximum length, it is favorable for avoiding the excessive contact between the skin and the conducting sheets while maintaining the effect of the conducting sheets so as to reduce the foreign body sensation when wearing the monitoring auxiliary device. Moreover, the following condition can be satisfied: 2.50 cm≤LCmax. Further, the following condition can be satisfied: 3.00 cm≤LCmax. Moreover, the following condition can be satisfied: 3.50 cm≤LCmax≤6.00 cm. Further, the following condition can be satisfied: 4.00 cm≤LCmax≤5.00 cm.

When a maximum interval between the at least two electrode points is EDmax, the following condition is satisfied: EDmax≤5.00 cm. Thus, by limiting the maximum interval between the electrode points, it is favorable for ensuring the distance between the electrode points to be closer, and for connecting to the monitoring device in smaller size so as to reduce the size of the monitoring device. Moreover, the following condition can be satisfied: EDmax≤4.00 cm. Further, the following condition can be satisfied: EDmax≤3.30 cm. Moreover, the following condition can be satisfied: EDmax≤2.80 cm. Further, the following condition can be satisfied: EDmax≤2.20 cm. Moreover, the following condition can be satisfied: 0 cm≤EDmax≤1.80 cm. Further, the following condition can be satisfied: 0.50 cm≤EDmax≤1.20 cm.

When the maximum interval between the at least two electrode points is EDmax, and the monitoring auxiliary device width of the monitoring auxiliary device is Wa, the following condition is satisfied: EDmax/Wa×10≤1.20. Thus, by limiting the ratio of the maximum interval between the electrode points and the monitoring auxiliary device width of the monitoring auxiliary device, it is favorable for reducing the size of the monitoring device connected to the electrode points so as to reduce the weight and volume of the device. Moreover, the following condition can be satisfied: EDmax/Wa×10≤1.10. Further, the following condition can be satisfied: 0≤EDmax/Wa×10≤0.90. Moreover, the following condition can be satisfied: 0.10≤EDmax/Wa×10≤0.80. Further, the following condition can be satisfied: 0.20≤EDmax/Wa×10≤0.50.

When a maximum interval between each of the electrode points and each of the conducting sheets is ECDmax, and the monitoring auxiliary device width of the monitoring auxiliary device is Wa, the following condition is satisfied: 0.10≤ECDmax/Wa. Therefore, it is favorable for ensuring the conducting sheets distributed widely and evenly on the monitoring auxiliary device by satisfying the ratio of the maximum interval between each of the electrode points and each of the conducting sheets and the monitoring auxiliary device width of the monitoring auxiliary device. Moreover, the following condition can be satisfied: 0.20≤ECDmax/Wa≤1.00. Further, the following condition can be satisfied: 0.30≤ECDmax/Wa≤0.80. Moreover, the following condition can be satisfied: 0.40≤ECDmax/Wa≤0.60. Further, the following condition can be satisfied: 0.42≤ECDmax/Wa≤0.50.

The monitoring auxiliary device can include an elastic band, and the conducting sheets overlap with the elastic band. The elastic band of the monitoring auxiliary device is favorable for attaching the specific area of the monitoring auxiliary device to the skin firmly, and increasing the tightness between the conducting sheets and the skin in the specific area so as to prevent the conducting sheets from apart the specific area.

The monitoring auxiliary device can further include an elastic fiber. The elastic fiber is favorable for clinging to skin, and increasing the tightness between the overall conducting sheets and the skin so as to reduce the failure of monitoring.

Each of the conducting sheets is in a directivity shape. Thus, by arranging the shape of the conducting sheets into a directional shape, it is favorable for improving the wearing recognition so as to help the user distinguish the wearing direction of the monitoring auxiliary device. In addition, the directivity shape of each of the conducting sheets is triangle, and it is favorable for further improving the wearing recognition.

The surface of each of the conducting sheets is a breathable structure, and the breathable structure is a mesh air vent. Thus, by providing the breathable structure with mesh air vent in the conducting sheets, it is favorable for increasing the breathable effect and avoiding the stuffiness on the user's skin.

The material of each of the conducting sheets includes a conductive polymer. Thus, by adding the conductive polymer into the conducting sheets, it is favorable for increasing the elasticity of the conducting sheets so as to prevent the conducting sheets from breaking due to twisting of the user's body.

The material of each of the conducting sheets includes graphene. Thus, by adding the graphene into the conducting sheets, it is favorable for increasing the breathable effect and thermal conductivity of the conducting sheets.

Each of the at least two conducting wires is a telescopic structure, and the telescopic structure is a polyline. Thus, by arranging the conducting wires into the polyline, it is favorable for increasing the ductility of the conducting wires so as to prevent the conducting wires from breaking due to excessive movement by the user.

Each of the conducting sheets and each of the conducting wires are detachable. Thus, by designing the conducting sheets and conducting wires in detachable, it is favorable for the monitoring auxiliary device being cleaned and reused so as to improving the efficiency of the monitoring auxiliary device.

The electrode points are located on the opposite sides of among of the conducting sheets. Thus, by arranging the electrode points and the conducting sheets on the opposite sides, while the conducting sheets are in contact with the skin, the electrode points are located on the opposite side of the skin contact surface. Therefore, it is favorable for avoiding the effect of wearing the monitoring auxiliary device when the monitoring device is connected to the electrode points so as to improve wearing comfort.

Each of the electrode points is a snap fastener. Thus, by arranging the electrode points into the snap fastener, it is favorable for making the connection between the electrode points and the monitoring device stronger so as to prevent the monitoring device from falling off.

Each of the aforementioned technical features of the monitoring auxiliary device can be combined with each other and reach the corresponded effect.

The monitoring auxiliary device of the present disclosure can assist the monitoring device to perform physiological monitoring. The physiological monitoring categories assisted by the monitoring auxiliary device can include sleeping, blood sugar, blood oxygen, blood pressure, temperature, respiration, heartbeat, electrocardiogram (ECG) and nerve impulses, etc. The monitoring auxiliary device can be a sleep monitoring auxiliary device, a blood glucose monitoring auxiliary device, a blood oxygen monitoring auxiliary device, a blood pressure monitoring auxiliary device, a temperature monitoring auxiliary device, a respiratory monitoring auxiliary device, a heartbeat monitoring auxiliary device, an ECG monitoring auxiliary device or a nerve impulse monitoring auxiliary device. The monitoring auxiliary device can be divided into the outer surface layer, the inner surface layer and the middle layer, which located between the outer surface layer and the inner surface layer. The inner side of the monitoring auxiliary device is the side closest to the user's skin while wearing, and the outer side of the monitoring auxiliary device is the side away from the user's skin while wearing.

The component of the monitoring auxiliary device of the present disclosure can include the electrode points, the conducting wires and the conducting sheets, and each of the electrode points, the conducting wires and the conducting sheets has conductive effects.

The component of the monitoring auxiliary device of the present disclosure can be arranged on the outer surface layer, the inner surface layer and the middle layer. For example, the electrode points can be disposed on the outer surface layer, the inner surface layer and the middle layer, the conducting wires can be disposed on the outer surface layer, the inner surface layer and the middle layer, and the conducting sheets can be disposed on the outer surface layer, the inner surface layer and the middle layer. The setting positions of the electrode points and the conducting sheets are determined according to their exposed positions. If the electrode points and the conducting sheets are exposed on the outer surface layer, the electrode points and the conducting sheets are regarded as disposed on the outer surface layer. If the electrode points and the conducting sheets are exposed on the inner surface layer, the electrode points and the conducting sheets are regarded as disposed on the inner surface layer. If the electrode points and the conducting sheets are not exposed, the electrode points and the conducting sheets are regarded as disposed on the middle layer. The setting positions of the conducting wires are determined according to where the largest volume of the conducting wires is located. For example, if 51% of the volume of the conducting wires is neither exposed on the outer surface layer nor exposed on the inner surface layer, the conducting wires are regarded as disposed on the middle layer. The aforementioned outer surface layer refers to the surface layer towards outside of the monitoring auxiliary device, the inner surface layer refers to the surface layer towards inside of the monitoring auxiliary device, and the middle layer refers to the non-exposed layer between the outer surface layer and the inner surface layer.

The monitoring auxiliary device can include at least one electrode point, at least two electrode points, at least three electrode points, at least four electrode points or at least five electrode points. The monitoring auxiliary device can further include only two electrode points, only three electrode points, only four electrode points or only five electrode points. The monitoring auxiliary device can include at least one conducting wire, at least two conducting wires, at least three conducting wires, at least four conducting wires or at least five conducting wires. The monitoring auxiliary device can further include only two conducting wires, only three conducting wires, only four conducting wires or only five conducting wires. The monitoring auxiliary device can include at least one conducting sheet, at least two conducting sheets, at least three conducting sheets, at least four conducting sheets or at least five conducting sheets. The monitoring auxiliary device can further include only two conducting sheets, only three conducting sheets, only four conducting sheets or only five conducting sheets.

The type of the monitoring auxiliary device of the present disclosure can be a wearable item. The wearable item can include an upper limb wearable item and a lower limb wearable item, the upper limb wearable item can be hat, headphone, neckband, collar, neck ring, cloth, outerwear, pajamas, bra, bust reducer, armband, sleeve, bracelet, watchband or watch, etc.; the lower limb wearable item can be corset, belt, pant, outer pant, pajamas, underwear, knee pad, ankle pad, anklet or socks, etc. The monitoring auxiliary device can further be composed of at least one wearable item, at least two wearable items, at least three wearable items, or at least four wearable items. For example, the monitoring auxiliary device can be sleeves and bracelets worn on both hands, anklets or socks worn on both feet.

The material of the monitoring auxiliary device of the present disclosure can include elastic fiber, and the elastic fiber is configured to increase the tightness between the overall conducting sheets and the skin. The material of the monitoring auxiliary device of the present disclosure can include the elastic band, and positions of the elastic band and the conducting sheets can overlap with each other so as to increase the tightness between the conducting sheets and the skin in a specific area. Elastic fiber refers to a material with high elongation at break, and the common elastic fibers include polyurethane fiber (spandex), diene elastic fiber (rubber yarn), polyetherester elastic fiber, natural rubber and synthetic rubber, etc.

The monitoring auxiliary device width of the present disclosure refers to the size of the monitoring auxiliary device. When the monitoring auxiliary device is the upper limb wearable item, the monitoring auxiliary device width is a length of the monitoring auxiliary device at 50% of the clothing. The length of monitoring auxiliary device at 50% of the clothing refers to the width of the monitoring auxiliary device laid flat and not stretched. If the length of the monitoring auxiliary device at 50% of the clothing cannot be measured, the width of the largest opening of the monitoring auxiliary device will be taken as the monitoring auxiliary device width. For example, the width of a bra cannot be measured because 50% of the bra is the hollow, and it will be regard as the length cannot be measured at 50% of the clothing. When the monitoring auxiliary device is the lower limb wearable item, the monitoring auxiliary device width is a waist width of the monitoring auxiliary device. The waist width of the monitoring auxiliary device refers to the width of the monitoring auxiliary device laid flat and not stretched. If the waist width of the monitoring auxiliary device cannot be measured, the width of the largest opening of the monitoring auxiliary device will be taken as the monitoring auxiliary device width. The aforementioned “laid flat” refers to spreading the monitoring auxiliary device and laying it flat. If the monitoring auxiliary device has an elastic thickness, the width of the monitoring auxiliary device in the flattened state will be regard as the monitoring auxiliary device width. Further, if the width of the monitoring auxiliary device can be adjusted according to the user, the monitoring auxiliary device width will be measured in the actual state when worn by the user.

The electrode points of the monitoring auxiliary device of the present disclosure can be connected to the monitoring device and can be detachable. The aforementioned “detachable” refers to the components can be separated from the monitoring auxiliary device, and the separated components can be used normally after being reassembled with the monitoring auxiliary device. The electrode points can include at least one snap fastener, at least two snap fasteners or at least three snap fasteners. The aforementioned snap fastener refers to the snap fastener with protrusion structure or depression structure. The monitoring device can arrange the corresponding snap fasteners to make the connection between the electrode points and the monitoring device being more secure.

The interval between the electrode points of the present disclosure refers to the shortest distance between any two electrode points. The interval between the electrode points includes a first electrode point interval, a second electrode point interval and a third electrode point interval, and so on. The shortest distance between any two electrode points refers to the minimum straight-line distance on the surface of the monitoring auxiliary device. When the two electrode points are on the opposite side, take an edge point of one of the electrode point perpendicular to the surface of the monitoring auxiliary device to take a normal line, use the normal line to make projection points to the opposite side, connect all the projection points to form a contralateral projection, and calculate the shortest distance based on the position of the contralateral projection. The same side and the opposite side are distinguished according to the inner surface layer and the outer surface layer of the monitoring auxiliary device. The inner surface layer of the monitoring auxiliary device is the side closest to the user's skin while wearing, and the outer surface layer of the monitoring auxiliary device is the side away from the user's skin while wearing.

The maximum interval between the electrode points of the present disclosure refers to the maximum value from the interval between each of the electrode points.

The conducting wires of the monitoring auxiliary device of the present disclosure are configured to connect the electrode points and the conducting wires, and the conducting wires can be detachable. The aforementioned “detachable” refers to the components can be separated from the monitoring auxiliary device, and the separated components can be used normally after being reassembled with the monitoring auxiliary device. The conducting wires can be a telescopic structure, and the telescopic structure can be a polyline, a bend line, a winding wire, or the combination thereof.

The conducting sheets of the monitoring auxiliary device of the present disclosure can be contacted with the user's skin and can be detachable. The aforementioned “detachable” refers to the components can be separated from the monitoring auxiliary device, and the separated components can be used normally after being reassembled with the monitoring auxiliary device. The surface of each of the conducting sheets can include a breathable structure, and the breathable structure is configured to increase the breathable effect. The breathable structure can be a rectangular air vent, an oblique air vent, a mesh air vent, or the combination thereof, and the air vent refers to an opening structure.

The material of each of the conducting sheets can include a conductive polymer, metal, metal fiber, metal oxide, carbon black, carbon nanotube, graphite, graphene and carbon fiber. The conductive polymer can include polyacetylene, polyaniline, polypyrrole, polythiophen, polyphenylene sulfide and polystyrene. The metal fiber can include silver fiber and copper fiber.

The types of the conducting sheets of the present disclosure can be dry conducting sheets or wet conducting sheets. The dry conducting sheets refer to the conducting sheets that can conduct electricity normally without conducting glue, and the wet conducting sheets refer to the conducting sheets that require conducting glue to conduct electricity normally.

The shape of the conducting sheets of the present disclosure can be in a directivity shape to help the user distinguish the wearing direction of the monitoring auxiliary device. The directivity shape refers to the shape with directionality. Specifically, the directivity shape can be ellipse, triangle, polygon, petal-shaped, star-shaped, text, letter, arrow, irregular shape, or the combination thereof.

The maximum length of the conducting sheets of the present disclosure refers to the maximum straight-line distance which can be found on the plane of the conducting sheet. For example, depending on the shape of the conductive sheet, the maximum length of the conductive sheet can be the diameter of a circle, the long axis of an ellipse, the diagonal of a square or the longer diagonal of a rectangle.

The interval between the conducting sheets of the present disclosure refers to the shortest distance between any two conducting sheets. The interval between the conducting sheets includes a first conducting sheet interval, a second conducting sheet interval and a third conducting sheet interval, and so on. The shortest distance between any two conducting sheets refers to the minimum straight-line distance on the surface of the monitoring auxiliary device. When the two conducting sheets are on two sides being opposite, a normal line perpendicular to the surface of the monitoring auxiliary device is taken from an edge point of one of the conducting sheet, and the normal line can be used to make projection points to the opposite side. All of the projection points can be connected to form a contralateral projection, and the shortest distance can be calculated based on the position of the contralateral projection. The two conducting sheets are on the same side or the two sides being opposite are distinguished according to the inner surface layer and the outer surface layer of the monitoring auxiliary device. The inner surface layer of the monitoring auxiliary device is the side closest to the user's skin while wearing, and the outer surface layer of the monitoring auxiliary device is the side away from the user's skin while wearing.

The maximum interval between the conducting sheets of the present disclosure refers to the maximum value from the interval between each of the conducting sheets.

The interval between the electrode point and the conducting sheet refers to the shortest distance of the conducting wire connect the electrode point and the conducting sheet. The interval between the electrode point and the conducting sheet includes a first electrode point and conducting sheet interval, a second electrode point and conducting sheet interval and a third electrode point and conducting sheet interval, and so on. The shortest distance between the electrode point and the conducting sheet refers to the minimum straight-line distance on the surface of the monitoring auxiliary device. When the electrode point and the conducting sheet are on two sides being opposite, a normal line perpendicular to the surface of the monitoring auxiliary device is taken from an edge point of one of the electrode point or the conducting sheet, and the normal line can be used to make projection points to the opposite side. All of the projection points can be connected to form a contralateral projection, and the shortest distance can be calculated based on the position of the contralateral projection. The electrode point and the conducting sheet are on the same side or the two sides being opposite are distinguished according to the inner surface layer and the outer surface layer of the monitoring auxiliary device. The inner surface layer of the monitoring auxiliary device is the side closest to the user's skin, and the outer surface layer of the monitoring auxiliary device is the side away from the user's skin.

The maximum interval between the electrode point and the conducting sheet refers to the maximum value from the interval between each of the electrode points and each of the conducting sheets.

The monitoring device of the present disclosure is configured to monitor physiological functions and estimate physiological diseases. The physiological functions can include sleeping, blood sugar, blood oxygen, blood pressure, temperature, respiration, heartbeat, ECG and nerve impulses, etc. The physiological diseases can include insomnia, diabetes, asthma, wheezing, fever, hyperventilation, abnormal heart rhythm, hypertension, heart disease, atrioventricular fibrillation, myocardial infarction, aortic dissection and epilepsy, etc.

According to the above embodiment, specific examples are proposed below and explained in detail with the drawings. It should be noted that, for the convenience of illustrating the configuration, the perspective view of the present disclosure is a schematic diagram showing the horizontal relative position and shape of each component, and the top view of the present disclosure is a schematic diagram showing the internal and external relationship of each component. That is, the component thickness, the structural size and the structural shape are not completely in accordance with the actual scale, clearly stated herein.

### 1st Embodiment

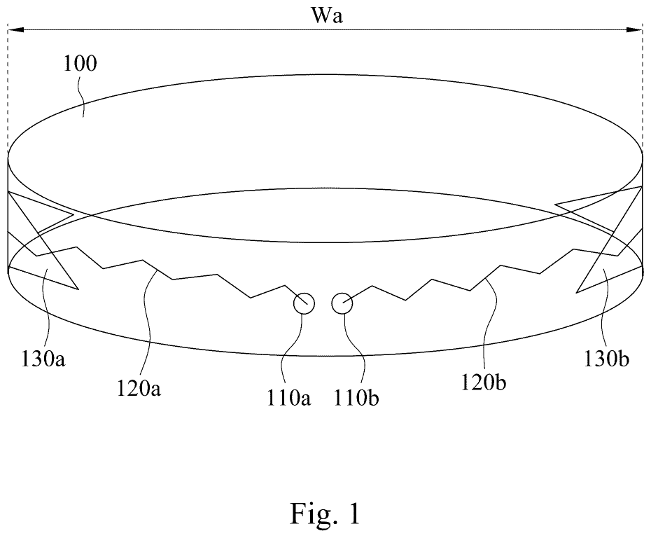

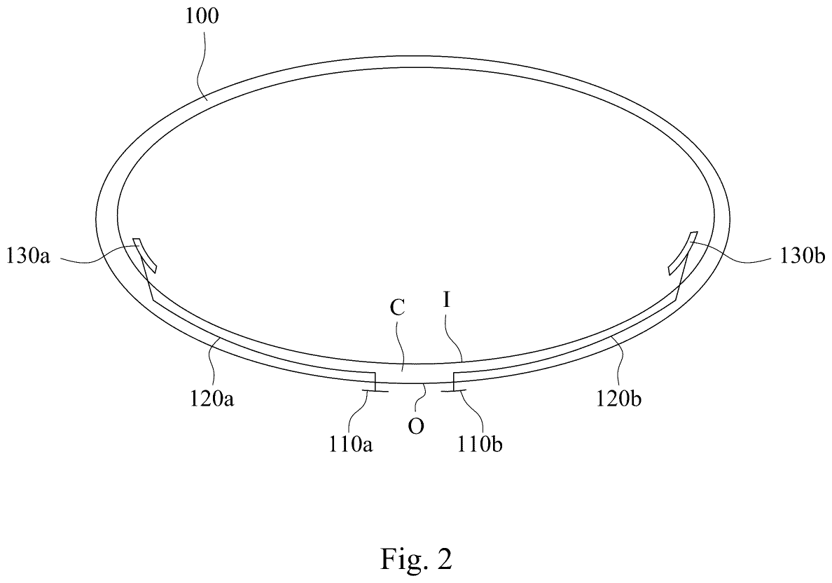

FIG. 1 is a perspective view of a monitoring auxiliary device 100 according to the 1st embodiment of the present disclosure. FIG. 2 is a top view of the monitoring auxiliary device 100 according to the 1st embodiment of FIG. 1. As shown in FIG. 1 and FIG. 2, in the 1 st embodiment, the monitoring auxiliary device 100 is a belt, and includes an outer surface layer O, an inner surface layer I, a middle layer C, two electrode points (first electrode point 110a, second electrode point 110b), two conducting wires (first conducting wire 120a, second conducting wire 120b) and two conducting sheets (first conducting sheet 130a, second conducting sheet 130b). The middle layer C is located between the outer surface layer O and the inner surface layer I. The inner surface layer I refers the side closest to the user's skin while wearing, the outer surface layer O refers the side away from the user's skin while wearing. The first electrode point 110a and the second electrode point 110b are disposed on the outer surface layer O, the first conducting sheet 130a and the second conducting sheet 130b are disposed on the inner surface layer I. The first conducting wire 120a is connected to the first electrode point 110a and the first conducting sheet 130a, the second conducting wire 120b is connected to the second electrode point 110b and the second conducting sheet 130b.

In the 1st embodiment, when an interval between the first electrode point 110a and the second electrode point 110b is a first electrode interval ED1 (its reference numeral is omitted), a maximum interval between the electrode points is EDmax, the following conditions are satisfied: ED1=3.50 cm; and EDmax=3.50 cm.

In the 1 st embodiment, the structure of the first conducting wire 120a and the structure of the second conducting wire 120b are polylines; the shape of the first conducting sheet 130a and the shape of the second conducting sheet 130b are triangles.

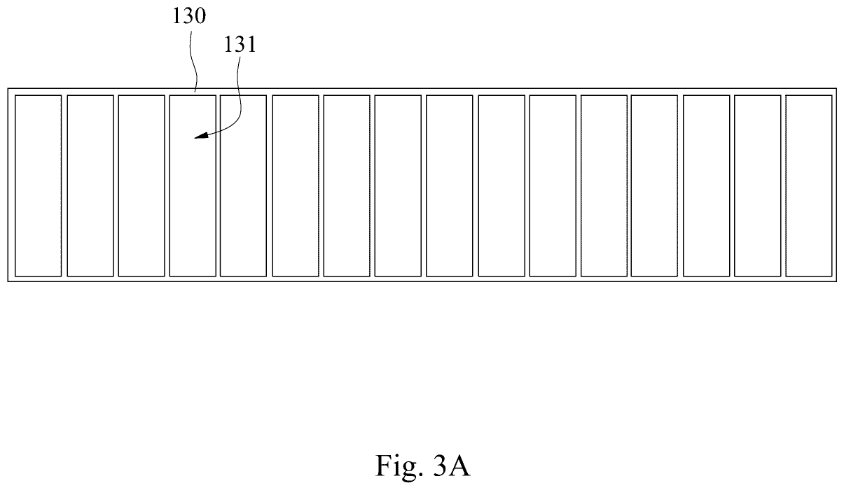

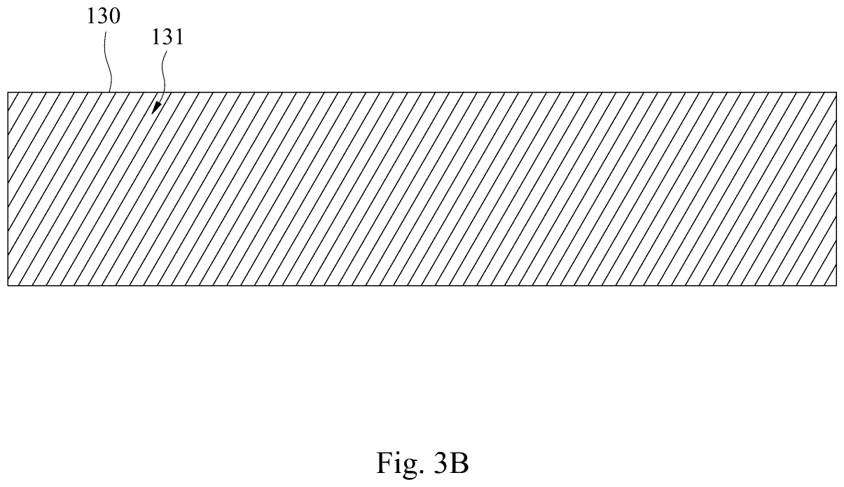

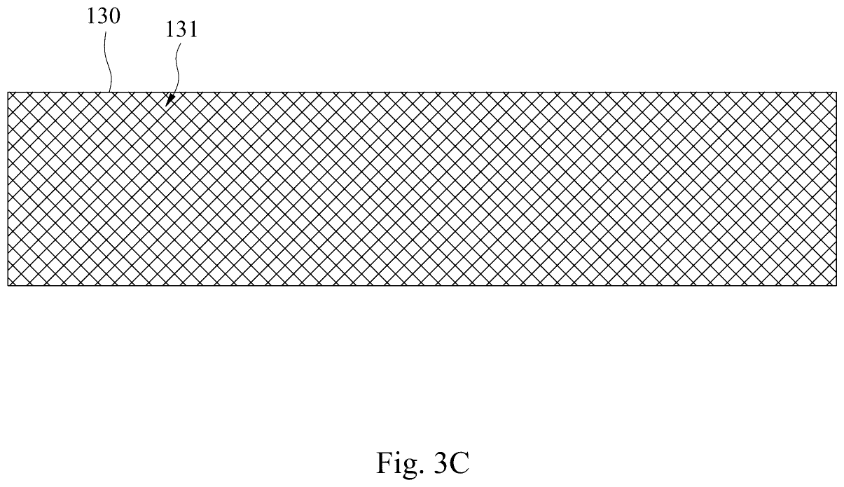

FIG. 3A is a schematic view of a breathable structure 131 of the conducting sheet 130 being a rectangular air vent. FIG. 3B is a schematic view of a breathable structure 131 of the conducting sheet 130 being an oblique air vent. FIG. 3C is a schematic view of a breathable structure 131 of the conducting sheet 130 being a mesh air vent. As shown in FIG. 1 to FIG. 3C, the surface of each of the conducting sheets 130 (the first conducting sheet 130a and the second conducting sheet 130b in FIG. 1 to FIG. 2) is a breathable structure 131, the breathable structure 131 is configured to increase the breathable effect. The breathable structure 131 can be the rectangular air vent, the oblique air vent, the mesh air vent, or the combination thereof, and the air vent refers to an opening structure. In FIG. 3A, the breathable structure 131 is the rectangular air vent; in FIG. 3B, the breathable structure 131 is the oblique air vent; in FIG. 3C, the breathable structure 131 is the mesh air vent.

In the 1 st embodiment, when a maximum length of the conducting sheets is LCmax, and the maximum length LCmax is the length of the longest side of the triangle, an interval between the first conducting sheet 130a and the second conducting sheet 130b is a first conducting sheet interval CD1 (its reference numeral is omitted), and a maximum interval between the conducting sheets is CDmax, the following conditions are satisfied: LCmax=3.00 cm; CD1=35.00 cm; and CDmax=35.00 cm.

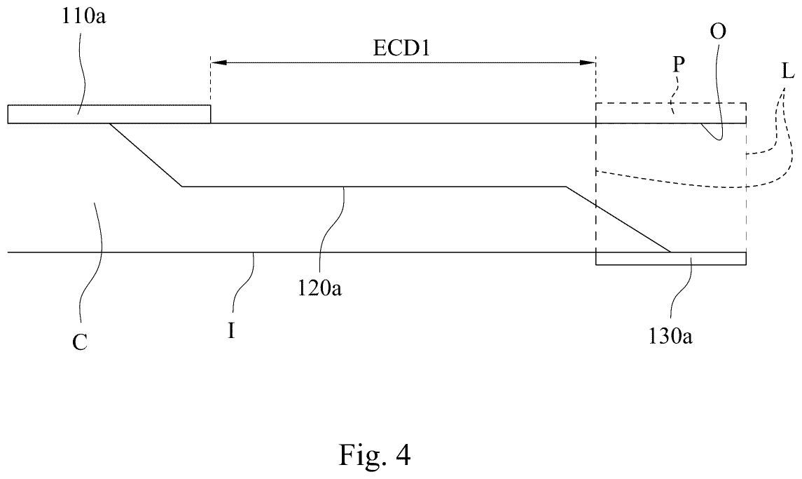

FIG. 4 is a schematic view of a first electrode point and conducting sheet interval ECD1 of the 1 st embodiment of FIG. 1. As shown in FIG. 1 and FIG. 4, the shortest distance between the first electrode point 110a and the first conducting sheet 130a connected by the first conducting wire 120a is the first electrode point and conducting sheet interval ECD1. In the 1st embodiment, since the first electrode point 110a and the first conducting sheet 130a are disposed on the outer surface O and the inner surface layer I respectively, and on two sides being opposite, a normal line L perpendicular to the outer surface O and the inner surface layer I of the monitoring auxiliary device 100 is taken from an edge point of the first conducting sheet 130a. The normal line L are used to make projection points to the opposite side, all of the projection points are connected to form a contralateral projection P, and the first electrode point and conducting sheet interval ECD1 is calculated based on the position of the contralateral projection P.

In the 1st embodiment, when the interval between the first electrode point 110a and the first conducting sheet 130a is the first electrode point and conducting sheet interval ECD1, an interval between the second electrode point 110b and the second conducting sheet 130b is a second electrode point and conducting sheet interval ECD2 (its reference numeral is omitted), and a maximum interval between each of the electrode points and each of the conducting sheets is ECDmax, the following conditions are satisfied: ECD1=15.75 cm; ECD2=15.75 cm; and ECDmax=15.75 cm.

In the 1st embodiment, the sleeping monitoring auxiliary device 100 is a lower limb wearable item, when a monitoring auxiliary device width Wa is a waist width of the monitoring auxiliary device 100, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 1.

TABLE 1

| | | | |
|---|---|---|---|
| Wa(cm) | 35.00 | EDmax/Wa × 10 | 1.00 |
| EDmax(cm) | 3.50 | LCmax/Wa | 0.09 |
| LCmax(cm) | 3.00 | CDmax/Wa | 1.00 |
| CDmax(cm) | 35.00 | ECDmax/Wa | 0.45 |
| ECDmax(cm) | 15.75 | | |

By limiting the ratio of the maximum interval between the electrode points and the monitoring auxiliary device width of the monitoring auxiliary device (EDmax/Wa×10), it is favorable for reducing the size of the monitoring device connected to the electrode points so as to reduce the weight and volume of the device. Thus, by designing the appropriate ratio of the maximum length of the conducting sheets and the monitoring auxiliary device width of the monitoring auxiliary device (LCmax/Wa), it is favorable for ensuring each of the conducting sheets with the sufficient proportion in the monitoring auxiliary device so as to increase the intensity of the potential signal. Thus, by satisfying the ratio of the maximum interval between the conducting sheets and the monitoring auxiliary device width of the monitoring auxiliary device (CDmax/Wa), it is favorable for providing enough distance between the conducting sheets to ensure the monitoring current flows widely through the body so as to improve the accuracy of the monitoring device. Thus, by satisfying the ratio of the maximum interval between each of the electrode points and each of the conducting sheets and the monitoring auxiliary device width of the monitoring auxiliary device (ECDmax/Wa), it is favorable for ensuring each of the conducting sheets distributed widely and evenly on the monitoring auxiliary device.

### 2nd Embodiment

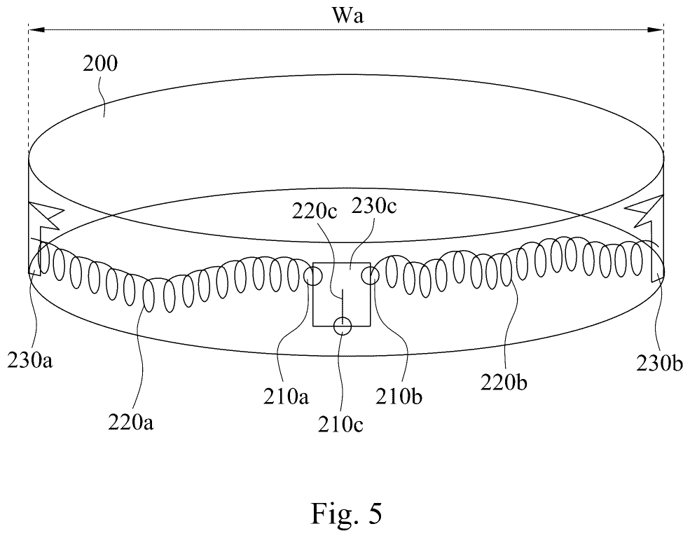

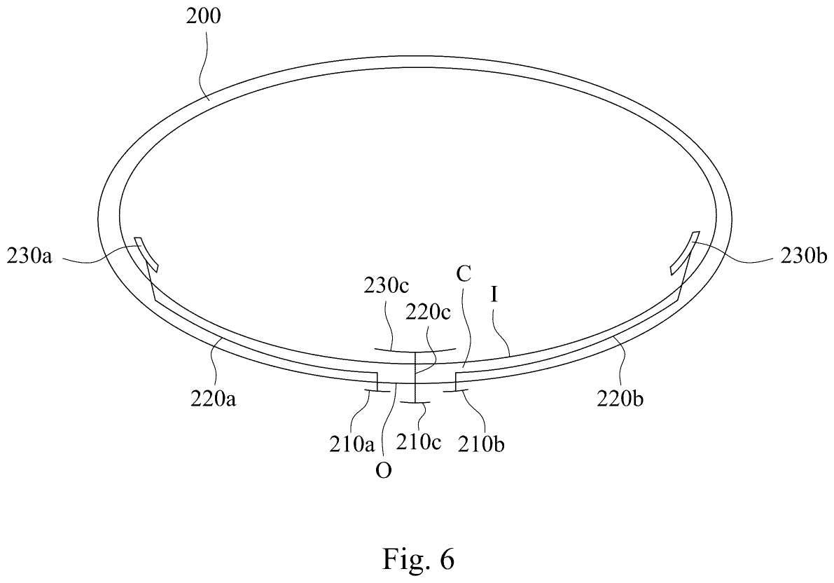

FIG. 5 is a perspective view of a monitoring auxiliary device 200 according to the 2nd embodiment of the present disclosure. FIG. 6 is a top view of the monitoring auxiliary device 200 according to the 2nd embodiment of FIG. 5. As shown in FIG. 5 and FIG. 6, in the 2nd embodiment, the monitoring auxiliary device 200 is a waist belt, and includes an outer surface layer O, an inner surface layer I, a middle layer C, three electrode points (first electrode point 210a, second electrode point 210b, third electrode point 210c), three conducting wires (first conducting wire 220a, second conducting wire 220b, third conducting wire 220c) and three conducting sheets (first conducting sheet 230a, second conducting sheet 230b, third conducting sheet 230c). In the 2nd embodiment, the first electrode point 210a, the second electrode point 210b and the third electrode point 210c are disposed on the outer surface layer O, the third electrode point 210c is located between the first electrode point 210a and the second electrode point 210b. The first conducting sheet 230a, the second conducting sheet 230b and the third conducting sheet 230c are disposed on the inner surface layer I, the third conducting sheet 230c is located between the first conducting sheet 230a and the second conducting sheet 230b. The first conducting wire 220a is connected to the first electrode point 210a and the first conducting sheet 230a, the second conducting wire 220b is connected to the second electrode point 210b and the second conducting sheet 230b, the third conducting wire 220c is connected to the third electrode point 210c and the third conducting sheet 230c. The configuration of the outer surface layer O, the inner surface layer I and the middle layer C are the same as the 1st embodiment, and will not be described again herein.

In the 2nd embodiment, when an interval between the first electrode point 210a and the third electrode point 210c is a first electrode interval ED1 (its reference numeral is omitted), an interval between the first electrode point 210a and the second electrode point 210b is a second electrode interval ED2 (its reference numeral is omitted), an interval between the second electrode point 210b and the third electrode point 210c is a third electrode interval ED3 (its reference numeral is omitted), and a maximum interval between the electrode points is EDmax, the following conditions are satisfied: ED1=1.50 cm; ED2=1.25 cm; ED3=1.50 cm; and EDmax=1.50 cm.

In the 2nd embodiment, the structure of the first conducting wire 220a, the structure of the second conducting wire 220b, and the structure of the third conducting wire 220c are winding wires; the shape of the first conducting sheet 230a, the shape of the second conducting sheet 230b and the shape of the third conducting sheet 230c are arrows.

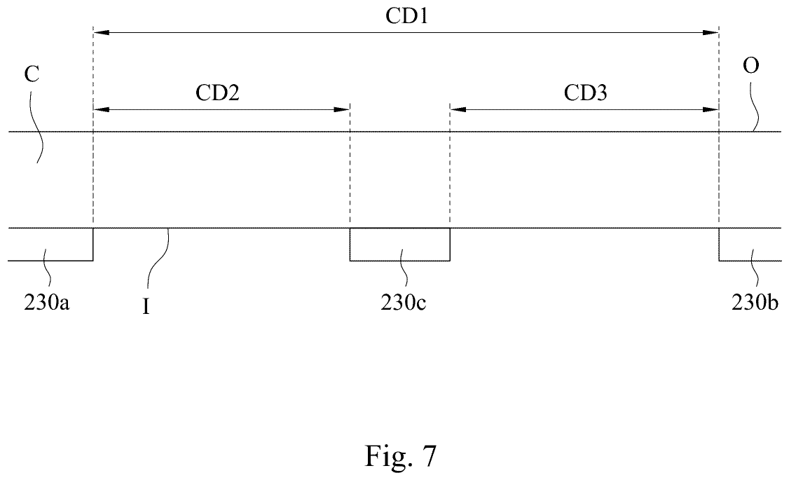

FIG. 7 is a schematic view of a first conducting sheet interval CD1, a second conducting sheet interval CD2 and a third conducting sheet interval CD3 according to the 2nd embodiment of FIG. 6. As shown in FIG. 5 to FIG. 7, in the 2nd embodiment, when a maximum length of the conducting sheets is LCmax, and the maximum length LCmax is the length of the arrow, an interval between the first conducting sheet 230a and the second conducting sheet 230b is a first conducting sheet interval CD1, an interval between the first conducting sheet 230a and the third conducting sheet 230c is a second conducting sheet interval CD2, an interval between the second conducting sheet 230b and the third conducting sheet 230c is a third conducting sheet interval CD3, and a maximum interval between the conducting sheets is CDmax, the following conditions are satisfied: LCmax=3.50 cm; CD1=30.00 cm; CD2=13.00 cm; CD3=13.00 cm; and CDmax=30.00 cm.

In the 2nd embodiment, when an interval between the first electrode point 210a and the first conducting sheet 230a is a first electrode point and conducting sheet interval ECD1 (its reference numeral is omitted), an interval between the second electrode point 210b and the second conducting sheet 230b is a second electrode point and conducting sheet interval ECD2 (its reference numeral is omitted), an interval between the third electrode point 210c and the third conducting sheet 230c is a third electrode point and conducting sheet interval ECD3, and a maximum interval between each of the electrode points and each of the conducting sheets is ECDmax, the following conditions are satisfied: 14.25 cm; ECD2=14.25 cm; CD3=0 cm; and ECDmax=14.25 cm.

It should be noted that, since the first electrode point 210a, the second electrode point 210b, the third electrode point 210c, and the first conducting sheet 230a, the second conducting sheet 230b, the third conducting sheet 230c are disposed on the outer surface O and the inner surface layer I, respectively. That is, located on the two sides being opposite, and the method of calculating the interval by projecting the normal line to the opposite side is the same as in the 1st embodiment, and will not be described again herein.

In the 2nd embodiment, the sleeping monitoring auxiliary device 200 is a lower limb wearable item, when a monitoring auxiliary device width Wa is a waist width of the monitoring auxiliary device 200, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 2.

TABLE 2

| | | | |
|---|---|---|---|
| Wa(cm) | 32.50 | EDmax/Wa × 10 | 0.46 |
| EDmax(cm) | 1.50 | LCmax/Wa | 0.11 |
| LCmax(cm) | 3.50 | CDmax/Wa | 0.92 |
| CDmax(cm) | 30.00 | ECDmax/Wa | 0.44 |
| ECDmax(cm) | 14.25 | | |

### 3rd Embodiment

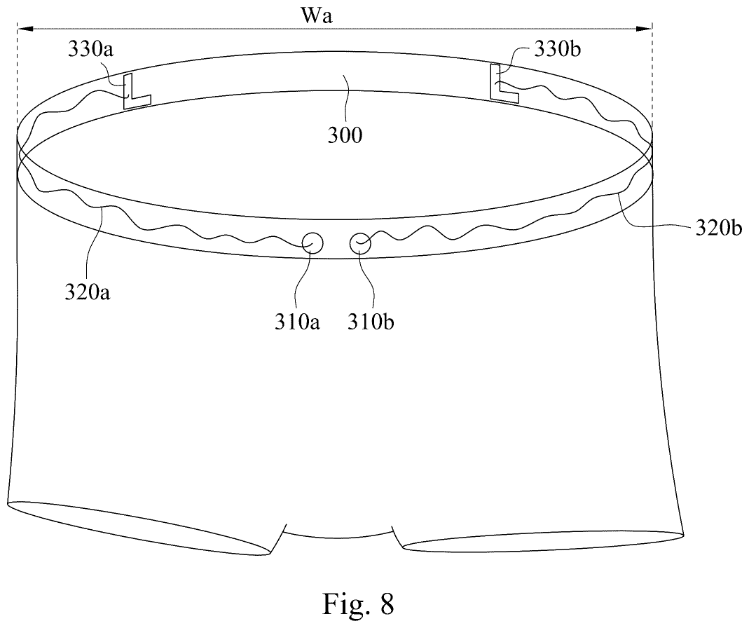

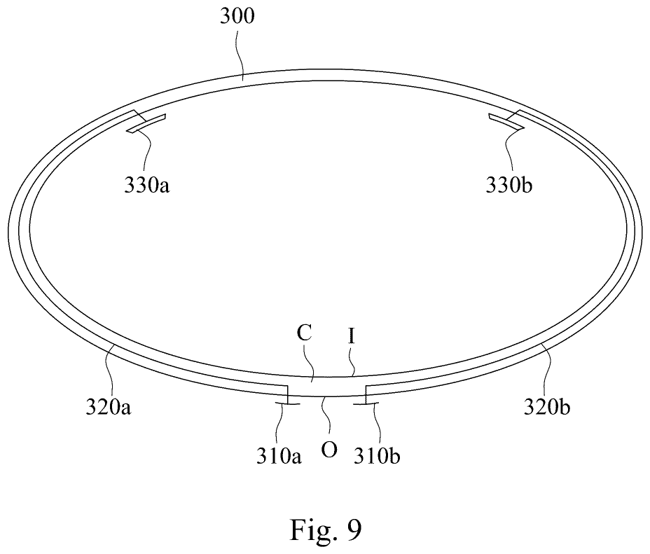

FIG. 8 is a perspective view of a monitoring auxiliary device 300 according to the 3rd embodiment of the present disclosure. FIG. 9 is a top view of the monitoring auxiliary device 300 according to the 3rd embodiment of FIG. 8. As shown in FIG. 8 and FIG. 9, in the 3rd embodiment, the monitoring auxiliary device 300 is an underwear and includes an outer surface layer O, an inner surface layer I, a middle layer C, two electrode points (first electrode point 310a, second electrode point 310b), two conducting wires (first conducting wire 320a, second conducting wire 320b) and two conducting sheets (first conducting sheet 330a, second conducting sheet 330b). The configuration of the outer surface layer O, the inner surface layer I, the middle layer C, the first electrode point 310a, the second electrode point 310b, the first conducting wire 320a, the second conducting wire 320b, the first conducting sheet 330a and the second conducting sheet 330b are the same as the 1st embodiment, and will not be described again herein.

In the 3rd embodiment, when an interval between the first electrode point 310a and the second electrode point 310b is a first electrode interval ED1 (its reference numeral is omitted), and a maximum interval between the electrode points is EDmax, the following conditions are satisfied: ED1=3.50 cm; and EDmax=3.50 cm.

In the 3rd embodiment, the structure of the first conducting wire 320a and the structure of the second conducting wire 320b are bend lines; the shape of the first conducting sheet 330a and the shape of the second conducting sheet 330b are in L shaped.

In the 3rd embodiment, when a maximum length of the conducting sheets is LCmax, and the maximum length LCmax is the length of the longest side of the L shaped, an interval between the first conducting sheet 330a and the second conducting sheet 330b is a first conducting sheet interval CD1 (its reference numeral is omitted), and a maximum interval between the conducting sheets is CDmax, the following conditions are satisfied: LCmax=4.00 cm; CD1=30.00 cm; and CDmax=30.00 cm.

In the 3rd embodiment, when the interval between the first electrode point 310a and the first conducting sheet 330a is the first electrode point and conducting sheet interval ECD1 (its reference numeral is omitted), an interval between the second electrode point 310b and the second conducting sheet 330b is a second electrode point and conducting sheet interval ECD2 (its reference numeral is omitted), and a maximum interval between each of the electrode points and each of the conducting sheets is ECDmax, the following conditions are satisfied: ECD1=13.25 cm; ECD2=13.25 cm; and ECDmax=13.25 cm.

It should be noted that, since the first electrode point 310a, the second electrode point 310b, and the first conducting sheet 330a, the second conducting sheet 330b are disposed on the outer surface O and the inner surface layer I, respectively. That is, located on the two sides being opposite, and the method of calculating the interval by projecting the normal line to the opposite side is the same as in the 1st embodiment, and will not be described again herein.

In the 3rd embodiment, the sleeping monitoring auxiliary device 300 is a lower limb wearable item, when a monitoring auxiliary device width Wa is a waist width of the monitoring auxiliary device 300, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 3.

TABLE 3

| | | | |
|---|---|---|---|
| Wa(cm) | 37.5 | EDmax/Wa × 10 | 0.93 |
| EDmax(cm) | 3.50 | LCmax/Wa | 0.11 |
| LCmax(cm) | 4.00 | CDmax/Wa | 0.80 |
| CDmax(cm) | 30.00 | ECDmax/Wa | 0.35 |
| ECDmax(cm) | 13.25 | | |

### 4th Embodiment

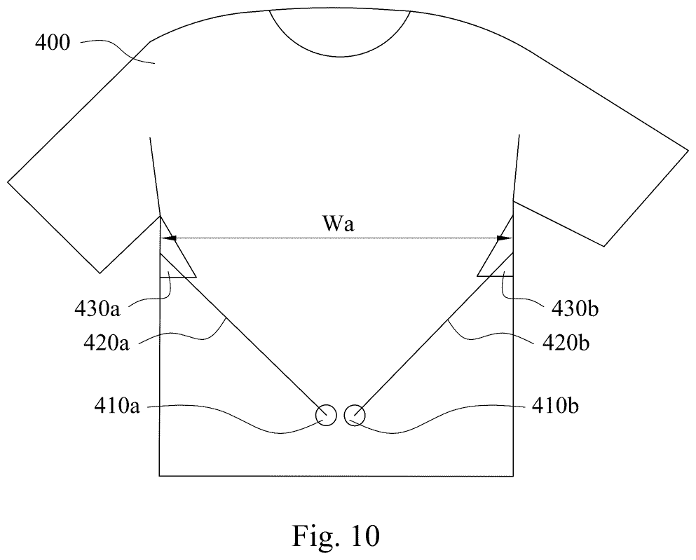

FIG. 10 is a perspective view of a monitoring auxiliary device 400 according to the 4th embodiment of the present disclosure. As shown in FIG. 10, in the 4th embodiment, the monitoring auxiliary device 400 is an outerwear and includes an outer surface layer, an inner surface layer, a middle layer, two electrode points (first electrode point 410a, second electrode point 410b), two conducting wires (first conducting wire 420a, second conducting wire 420b) and two conducting sheets (first conducting sheet 430a, second conducting sheet 430b). The first electrode point 410a and the second electrode point 410b are disposed on the outer surface layer, the first conducting sheet 430a and the second conducting sheet 430b are disposed on the inner surface layer. The configuration of the outer surface layer, the inner surface layer, the middle layer, the first electrode point 410a, the second electrode point 410b, the first conducting wire 420a, the second conducting wire 420b, the first conducting sheet 430a and the second conducting sheet 430b are the same as the 1st embodiment, and will not be described again herein.

In the 4th embodiment, when an interval between the first electrode point 410a and the second electrode point 410b is a first electrode interval ED1 (its reference numeral is omitted), and a maximum interval between the electrode points is EDmax, the following conditions are satisfied: ED1=3.00 cm; and EDmax=3.00 cm.

In the 4th embodiment, the structure of the first conducting wire 420a and the structure of the second conducting wire 420b are straight lines; the shape of the first conducting sheet 430a and the shape of the second conducting sheet 430b are triangles.

In the 4th embodiment, when a maximum length of the conducting sheets is LCmax, and the maximum length LCmax is the length of the longest side of the triangle, an interval between the first conducting sheet 430a and the second conducting sheet 430b is a first conducting sheet interval CD1 (its reference numeral is omitted), and a maximum interval between the conducting sheets is CDmax, the following conditions are satisfied: LCmax=5.00 cm; CD1=60.00 cm; and CDmax=60.00 cm.

In the 4th embodiment, when the interval between the first electrode point 410a and the first conducting sheet 430a is the first electrode point and conducting sheet interval ECD1 (its reference numeral is omitted), an interval between the second electrode point 410b and the second conducting sheet 430b is a second electrode point and conducting sheet interval ECD2 (its reference numeral is omitted), and a maximum interval between each of the electrode points and each of the conducting sheets is ECDmax, the following conditions are satisfied: ECD1=28.50 cm; ECD2=28.50 cm; and ECDmax=28.50 cm.

It should be noted that, since the first electrode point 410a, the second electrode point 410b, and the first conducting sheet 430a, the second conducting sheet 430b are disposed on the outer surface and the inner surface layer, respectively. That is, located on the two sides being opposite, and the method of calculating the interval by projecting the normal line to the opposite side is the same as in the 1st embodiment, and will not be described again herein.

In the 4th embodiment, the sleeping monitoring auxiliary device 400 is an upper limb wearable item, when a monitoring auxiliary device width Wa is a length of the monitoring auxiliary device 400 at 50% of the clothing, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 4.

TABLE 4

| | | | |
|---|---|---|---|
| Wa(cm) | 45.00 | EDmax/Wa × 10 | 0.67 |
| EDmax(cm) | 3.00 | LCmax/Wa | 0.11 |
| LCmax(cm) | 5.00 | CDmax/Wa | 1.33 |
| CDmax(cm) | 60.00 | ECDmax/Wa | 0.63 |
| ECDmax(cm) | 28.50 | | |

### 5th Embodiment

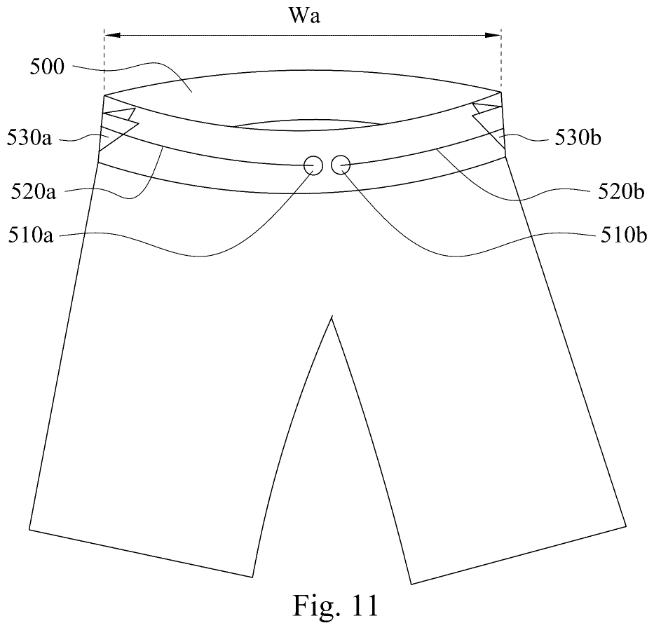

FIG. 11 is a perspective view of a monitoring auxiliary device 500 according to the 5th embodiment of the present disclosure. As shown in FIG. 11, in the 5th embodiment, the monitoring auxiliary device 500 is an outerwear and includes an outer surface layer, an inner surface layer, a middle layer, two electrode points (first electrode point 510a, second electrode point 510b), two conducting wires (first conducting wire 520a, second conducting wire 520b) and two conducting sheets (first conducting sheet 530a, second conducting sheet 530b). The configuration of the outer surface layer, the inner surface layer, the middle layer, the first electrode point 510a, the second electrode point 510b, the first conducting wire 520a, the second conducting wire 520b, the first conducting sheet 530a and the second conducting sheet 530b are the same as the 1st embodiment, and will not be described again herein.

In the 5th embodiment, when an interval between the first electrode point 510a and the second electrode point 510b is a first electrode interval ED1 (its reference numeral is omitted), and a maximum interval between the electrode points is EDmax, the following conditions are satisfied: ED1=2.50 cm; and EDmax=2.50 cm.

In the 5th embodiment, the structure of the first conducting wire 520a, the structure of the second conducting wire 520b, the shape of the first conducting sheet 530a and the shape of the second conducting sheet 530b are the same as the 4th embodiment, and will not be described again herein.

In the 5th embodiment, when a maximum length of the conducting sheets is LCmax, and the maximum length LCmax is the length of the longest side of the triangle, an interval between the first conducting sheet 530a and the second conducting sheet 530b is a first conducting sheet interval CD1 (its reference numeral is omitted), and a maximum interval between the conducting sheets is CDmax, the following conditions are satisfied: LCmax=4.50 cm; CD1=39.00 cm; and CDmax=39.00 cm.

In the 5th embodiment, when the interval between the first electrode point 510a and the first conducting sheet 530a is the first electrode point and conducting sheet interval ECD1 (its reference numeral is omitted), an interval between the second electrode point 510b and the second conducting sheet 530b is a second electrode point and conducting sheet interval ECD2 (its reference numeral is omitted), and a maximum interval between each of the electrode points and each of the conducting sheets is ECDmax, the following conditions are satisfied: ECD1=18.25 cm; ECD2=18.25 cm; and ECDmax=18.25 cm.

It should be noted that, since the first electrode point 510a, the second electrode point 510b, and the first conducting sheet 530a, the second conducting sheet 530b are disposed on the outer surface and the inner surface layer, respectively. That is, located on the two sides being opposite, and the method of calculating the interval by projecting the normal line to the opposite side is the same as in the 1st embodiment, and will not be described again herein.

In the 5th embodiment, the sleeping monitoring auxiliary device 500 is an lower limb wearable item, when a monitoring auxiliary device width Wa is a waist width of the monitoring auxiliary device 500, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 5.

TABLE 5

| | | | |
|---|---|---|---|
| Wa(cm) | 40.00 | EDmax/Wa × 10 | 0.63 |
| EDmax(cm) | 2.50 | LCmax/Wa | 0.11 |
| LCmax(cm) | 4.50 | CDmax/Wa | 0.98 |
| CDmax(cm) | 39.00 | ECDmax/Wa | 0.46 |
| ECDmax(cm) | 18.25 | | |

### 6th Embodiment

In the 6th embodiment, the monitoring auxiliary device is a bra and includes an outer surface layer, an inner surface layer, a middle layer, three electrode points (first electrode point, second electrode point, third electrode point), three conducting wires (first conducting wire, second conducting wire, third conducting wire) and three conducting sheets (first conducting sheet, second conducting sheet, third conducting sheet). The configuration of the outer surface layer, the inner surface layer, the middle layer, the first electrode point, the second electrode point, the third electrode point, the first conducting wire, the second conducting wire, the third conducting wire, the first conducting sheet, the second conducting sheet and the third conducting sheet are the same as the 2nd embodiment, and will not be described again herein.

In the 6th embodiment, when an interval between the first electrode point and the third electrode point is a first electrode interval ED1, an interval between the first electrode point and the second electrode point is a second electrode interval ED2, an interval between the second electrode point and the third electrode point is a third electrode interval ED3, and a maximum interval between the electrode points is EDmax, the following conditions are satisfied: ED1=2.00 cm; ED2=1.50 cm; ED3=1.50 cm; and EDmax=2.00 cm.

In the 6th embodiment, the structure of the first conducting wire, the structure of the second conducting wire, the structure of the third conducting wire, the shape of the first conducting sheet, the shape of the second conducting sheet and the shape of the third conducting sheet are the same as the 4th embodiment, and will not be described again herein.

In the 6th embodiment, when a maximum length of the conducting sheets is LCmax, and the maximum length LCmax is the length of the longest side of the triangle, an interval between the first conducting sheet and the second conducting sheet is a first conducting sheet interval CD1, an interval between the first conducting sheet and the third conducting sheet is a second conducting sheet interval CD2, an interval between the second conducting sheet and the third conducting sheet is a third conducting sheet interval CD3, and a maximum interval between the conducting sheets is CDmax, the following conditions are satisfied: LCmax=4.00 cm; CD1=50.00 cm; CD2=22.00 cm; CD3=22.00 cm; and CDmax=50.00 cm.

In the 6th embodiment, when an interval between the first electrode point and the first conducting sheet is a first electrode point and conducting sheet interval ECD1, an interval between the second electrode point and the second conducting sheet is a second electrode point and conducting sheet interval ECD2, an interval between the third electrode point and the third conducting sheet is a third electrode point and conducting sheet interval ECD3, and a maximum interval between each of the electrode points and each of the conducting sheets is ECDmax, the following conditions are satisfied: ECD1=24.00 cm; ECD2=24.00 cm; ECD3=0 cm; and ECDmax=24.00 cm.

It should be noted that, since the first electrode point, the second electrode point, the third electrode point, the first conducting sheet, the second conducting sheet, the third conducting sheet are disposed on the outer surface and the inner surface layer, respectively. That is, located on the two sides being opposite, and the method of calculating the interval by projecting the normal line to the opposite side is the same as in the 1st embodiment, and will not be described again herein.

In the 6th embodiment, the sleeping monitoring auxiliary device is a upper limb wearable item, when a monitoring auxiliary device width Wa is the width of the largest opening of the monitoring auxiliary device, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 6.

TABLE 6

| | | | |
|---|---|---|---|
| Wa(cm) | 42.50 | EDmax/Wa × 10 | 0.47 |
| EDmax(cm) | 2.00 | LCmax/Wa | 0.09 |
| LCmax(cm) | 4.00 | CDmax/Wa | 1.18 |
| CDmax(cm) | 50.00 | ECDmax/Wa | 0.56 |
| ECDmax(cm) | 24.00 | | |

### 7th Embodiment

In the 7th embodiment, the monitoring auxiliary device is a pajamas and includes an outer surface layer, an inner surface layer, a middle layer, two electrode points (first electrode point, second electrode point), two conducting wires (first conducting wire, second conducting wire) and two conducting sheets (first conducting sheet, second conducting sheet). The configuration of the outer surface layer, the inner surface layer, the middle layer, the first electrode point, the second electrode point, the first conducting wire, the second conducting wire, the first conducting sheet and the second conducting sheet are the same as the 1st embodiment, and will not be described again herein.

In the 7th embodiment, when an interval between the first electrode point and the second electrode point is a first electrode interval ED1, and a maximum interval between the electrode points is EDmax, the following conditions are satisfied: ED1=1.00 cm; and EDmax=1.00 cm.

In the 7th embodiment, the structure of the first conducting wire, the structure of the second conducting wire, the shape of the first conducting sheet and the shape of the second conducting sheet are the same as the 4th embodiment, and will not be described again herein.

In the 7th embodiment, when a maximum length of the conducting sheets is LCmax, and the maximum length LCmax is the length of the longest side of the triangle, an interval between the first conducting sheet and the second conducting sheet is a first conducting sheet interval CD1, and a maximum interval between the conducting sheets is CDmax, the following conditions are satisfied: LCmax=5.00 cm; CD1=28.00 cm; and CDmax=28.00 cm.

In the 7th embodiment, when the interval between the first electrode point and the first conducting sheet is the first electrode point and conducting sheet interval ECD1, an interval between the second electrode point and the second conducting sheet is a second electrode point and conducting sheet interval ECD2, and a maximum interval between each of the electrode points and each of the conducting sheets is ECDmax, the following conditions are satisfied: ECD1=13.00 cm; ECD2=13.00 cm; and ECDmax=13.00 cm.

It should be noted that, since the first electrode point, the second electrode point, and the first conducting sheet, the second conducting sheet are disposed on the outer surface and the inner surface layer, respectively. That is, located on the two sides being opposite, and the method of calculating the interval by projecting the normal line to the opposite side is the same as in the 1st embodiment, and will not be described again herein.

In the 7th embodiment, the sleeping monitoring auxiliary device is an upper limb wearable item, when a monitoring auxiliary device width Wa is a length of the monitoring auxiliary device at 50% of the clothing, the maximum interval between the electrode points is EDmax, the maximum length of the conducting sheets is LCmax, the maximum interval between the conducting sheets is CDmax, the maximum interval between each of the electrode points and each of the conducting sheets is ECDmax, the conditions related to the parameters can be satisfied as the following Table 7.

TABLE 7

| | | | |
|---|---|---|---|
| Wa(cm) | 30.00 | EDmax/Wa × 10 | 0.33 |
| EDmax(cm) | 1.00 | LCmax/Wa | 0.17 |
| LCmax(cm) | 5.00 | CDmax/Wa | 0.93 |
| CDmax(cm) | 28.00 | ECDmax/Wa | 0.43 |
| ECDmax(cm) | 13.00 | | |

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A monitoring auxiliary device, which is a sleeping monitoring auxiliary device, the sleeping monitoring auxiliary device being a wearable item which is a lower limb wearable item, the monitoring auxiliary device comprising:
an outer surface layer;
an inner surface layer;
a middle layer located between the outer surface layer and the inner surface layer;
at least two electrode points comprising a first electrode point and a second electrode point;
at least two conducting wires comprising a first conducting wire and a second conducting wire; and
at least two conducting sheets comprising a first conducting sheet and a second conducting sheet;
wherein the at least two electrode points are disposed on the outer surface layer of the monitoring auxiliary device, and the at least two conducting sheets are disposed on the inner surface layer of the monitoring auxiliary device, the first conducting wire is connected to the first electrode point and the first conducting sheet, the second conducting wire is connected to the second electrode point and the second conducting sheet;
wherein a maximum interval between the at least two conducting sheets is CDmax, a monitoring auxiliary device width of the monitoring auxiliary device is Wa, and the following condition is satisfied:
$$0.6 \leq CDmax / Wa .$$

2. The monitoring auxiliary device of claim 1, wherein the maximum interval between the at least two conducting sheets is CDmax, and the following condition is satisfied:
$$15 cm \leq CDmax .$$

3. The monitoring auxiliary device of claim 1, wherein a maximum length of the at least two conducting sheets is LCmax, the monitoring auxiliary device width of the monitoring auxiliary device is Wa, and the following condition is satisfied:
$$0.05 \leq LCmax / Wa .$$

4. The monitoring auxiliary device of claim 3, wherein the maximum length of the at least two conducting sheets is LCmax, and the following condition is satisfied:
$$2.5 cm \leq LCmax \leq 5 cm .$$

5. The monitoring auxiliary device of claim 1, wherein a maximum interval between the at least two electrode points is EDmax, and the following condition is satisfied:
$$EDmax \leq 5. cm .$$

6. The monitoring auxiliary device of claim 3, wherein a maximum interval between the at least two electrode points is EDmax, the monitoring auxiliary device width of the monitoring auxiliary device is Wa, and the following condition is satisfied:
$$EDmax / Wa \times 10 \leq 1.2 .$$

7. The monitoring auxiliary device of claim 1, wherein a maximum interval between each of the at least two electrode points and each of the conducting sheets is ECDmax, the monitoring auxiliary device width of the monitoring auxiliary device is Wa, and the following condition is satisfied:
$$0.1 \leq ECDmax / Wa .$$

8. The monitoring auxiliary device of claim 1, further comprising an elastic band, and the at least two conducting sheets overlap with the elastic band.

9. The monitoring auxiliary device of claim 1, wherein each of the at least two conducting sheets is in a directivity shape.

10. The monitoring auxiliary device of claim 9, wherein the directivity shape of each of the at least two conducting sheets is triangle.

11. The monitoring auxiliary device of claim 1, wherein each of the at least two conducting wires is a telescopic structure, and the telescopic structure is a polyline.

12. A monitoring auxiliary device, which is a wearable item, the monitoring auxiliary device comprising:
at least two electrode points comprising a first electrode point and a second electrode point;
at least two conducting wires comprising a first conducting wire and a second conducting wire; and
at least two conducting sheets comprising a first conducting sheet and a second conducting sheet;
wherein the first conducting wire is connected to the first electrode point and the first conducting sheet, the second conducting wire is connected to the second electrode point and the second conducting sheet;
wherein a maximum interval between the at least two conducting sheets is CDmax, a monitoring auxiliary device width of the monitoring auxiliary device is Wa, and the following condition is satisfied:
$$0.7 \leq CDmax / Wa .$$

13. The monitoring auxiliary device of claim 12, wherein a maximum interval between the at least two electrode points is EDmax, the monitoring auxiliary device width of the monitoring auxiliary device is Wa, and the following condition is satisfied:
$$EDmax / Wa \times 10 \leq 1.1 .$$

14. The monitoring auxiliary device of claim 13, wherein a maximum interval between each of the at least two electrode points and each of the at least two conducting sheets is ECDmax, the monitoring auxiliary device width of the monitoring auxiliary device is Wa, and the following condition is satisfied:
$$0.2 \leq ECDmax / Wa \leq 1. .$$

15. The monitoring auxiliary device of claim 14, wherein the maximum interval between the at least two conducting sheets is CDmax, and the following condition is satisfied:
$$7.5 cm \leq CDmax .$$

16. The monitoring auxiliary device of claim 14, wherein the maximum interval between the at least two electrode points is EDmax, and the following condition is satisfied:
$$EDmax \leq 3.3 cm .$$

17. The monitoring auxiliary device of claim 16, wherein a surface of each of the at least two conducting sheets is a breathable structure, and the breathable structure is a mesh air vent.

18. The monitoring auxiliary device of claim 17, wherein a material of each of the at least two conducting sheets comprises a conductive polymer.

19. The monitoring auxiliary device of claim 17, wherein a material of each of the at least two conducting sheets comprises graphene.

20. The monitoring auxiliary device of claim 17, wherein among of the at least two electrode points are located on the opposite sides of among of the at least two conducting sheets.

21. The monitoring auxiliary device of claim 20, further comprising an elastic fiber.

22. The monitoring auxiliary device of claim 21, further comprising an elastic band, and the at least two conducting sheets overlap with the elastic band.

23. The monitoring auxiliary device of claim 22, wherein each of the at least two electrode points is a snap fastener.

24. The monitoring auxiliary device of claim 23, wherein each of the at least two conducting sheets and each of the at least two conducting wires are detachable.

25. The monitoring auxiliary device of claim 24, wherein,
the at least two electrode points further comprise a third electrode point;
the at least two conducting wires further comprise a third conducting wire; and
the at least two conducting sheets further comprise a third conducting sheet.
